(19) United States
(12) Patent Application Publication
(10) Pub. No.: US 2009/0318753 A1
(43) Pub. Date: Dec. 24, 2009

(54) Vibrator with Integrated Clock and Reminder System

(76) Inventor: Dany Antoun Metri, Miami, FL (US)

Correspondence Address: Dany Metri, 808 Brickell Key Drive #3302, Miami, FL, 33131, US

(21) Appl. No.: 12/437,184

(22) Filed: May 7, 2009

Related U.S. Application Data
(60) Provisional application No. 61/074,078, filed on Jun. 19, 2008.

(51) Int. Cl. A61F 5/00; A61H 1/00; G04B 47/00

(52) U.S. Cl. 600/38; 601/46; 368/10

(57) ABSTRACT

The present invention is a vibrator with an integrated alarm/reminder clock that initiates the vibration function of the vibrator for a predetermined period of time. The core components of the device are: a housing, a motor that powers a vibrating mechanism contained within the housing, an alarm clock/reminder system and a power source. The power source is preferably one or more batteries, and the alarm clock/reminder element is preferably digital. The invention can be used as a sex toy so as to keep the user's sex life healthy and active by setting and initiating the mood for sexual activity through a learned response to a reminder system/pattern of use/method of use a user develops by using the device and method.

### BACKGROUND OF THE INVENTION

1. Field of the Invention

The invention relates generally to a personal massage device used for sexual stimulation. More specifically, the invention relates to a vibrator with an integrated alarm clock/reminder system that operates at a preset time so as to initiate the vibration function and associated sexual stimulation.

2. Background

Currently there are a number of solutions available to encourage individuals to engage in sexual activities: Films, games, devices, clothing, oils, massagers, etc. Some of these solutions attempt to change the shape or the size of the personal massage devices/toys, but these solutions fail to meet the needs of the individual because generally is still the same concept; i.e., the user(s) needs to be in the mood to use devices/toys to engage in any subsequent sexual activity. In contrast, the present invention will set up the mood for the user with a reminder function. Other past solutions that attempt to use different materials and shear variety, i.e., like glass, or differently shaped/colored devices, likewise do not change past pattern(s) of use. These solutions are similarly unable to meet the needs of the industry/individual because it is still the same old fashioned way of use, i.e., sexual mood needing to precede actual use of the device regardless of circumstance.

As a result of past solution shortcomings, many physical relationships between individuals and, indeed, a person's own sexuality and desire for sexual activity can wane over time to an unhealthy and severely diminished threshold. Patterns of behavior develop over time that simply do not include time for, or interest in, sexual activity. Relationships suffer and personal perspectives can be permanently altered in negative ways. Individual's lives can unintentionally be reconstituted so as to include no sexual activity what-so-ever.

### SUMMARY OF THE INVENTION

The present invention is a vibrator/personal massager having an integrated alarm clock/reminder system. The basic device includes a vibrating element, a power source, and a housing that is preferably made of either plastic or silicone and/or includes provision for receiving a replaceable cover(s) of various sizes and textures. These elements are connected as follows: a printed circuit board (“Pcb”) is connected between the alarm clock and the first battery, to power up the reminder/clock/alarm function, and then another Pcb is connected between the first battery and the other batteries and they are connected to a motor through a battery spring and motor leads.

It is an object of the present invention to provide a device that wakes the user up (if they are sleeping) and simultaneously provides a reminder and encouragement to engage in sexual activities, either together with a partner or alone, as may be the case. After several uses, a pattern of conduct will emerge wherein the necessary preceding mood is set-up by the initiation of the vibrator operation and/or the accompanying sound of a brief alarm tone (i.e., ringing, musical chime, song clip, voice over, etc.) It is an object of the present invention to provide a vibrator with an integrated alarm/reminder function that does not suffer from any of the problems or deficiencies associated with prior solutions where use is only preceded by a necessary mood and where mood creation is separate and apart from the device itself.

The present invention now will be described more fully hereinafter with reference to the accompanying drawings, which are intended to be read in conjunction with both this summary, the detailed description and any preferred and/or particular embodiments specifically discussed or otherwise disclosed. This invention may, however, be embodied in many different forms and should not be construed as limited to the embodiments set forth herein; rather, these embodiments are provided by way of illustration only and so that this disclosure will be thorough, complete and will fully convey the full scope of the invention to those skilled in the art.

### BRIEF DESCRIPTION OF THE DRAWINGS

FIG. 1 is an illustration of the vibrator equipped with an optional cover.

FIG. 2 is a schematic illustration of the vibrator.

FIG. 3 is an illustration of the vibrator equipped with an alternate cover.

FIG. 4 is an illustration of a schematic of an alternate embodiment of the vibrator.

FIG. 5 is a sectional schematic of the vibrator according to the present invention.

FIG. 6 is a cut away showing the inserted sheath of the vibrator of the present invention.

FIG. 7 shows an alarm clock suitable for use with the present invention.

### DETAILED DESCRIPTION OF THE INVENTION

The present invention is directed to a vibrator with an integrated alarm/reminder function.

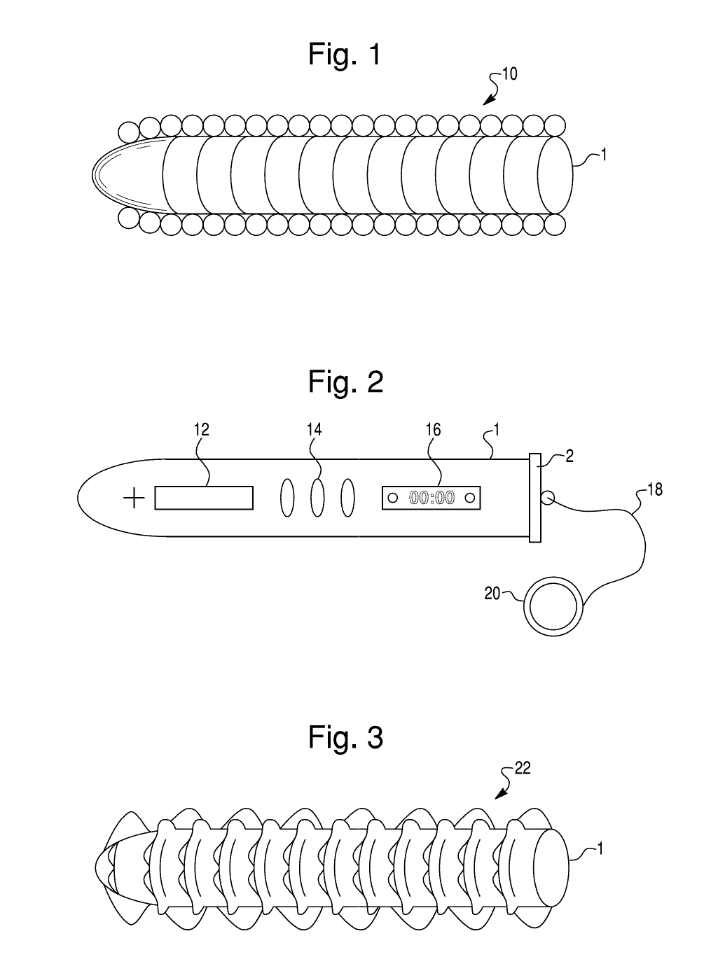

FIGS. 1 and 3 show various covers 10 and 22 to cover the housing 1, wherein the housing slides inside the cover(s) 10 and 22. In the FIG. 1 embodiment, the cover 10 is covered by little balls (the cover 10 and the little balls are integrated). The cover 10 is an accessory to the housing 1 and an option for the user to get a different feeling and touch. The cover 10 could be made in different shapes or models instead of little balls it could be little stars, rib elements, etc., as shown in FIG. 3.

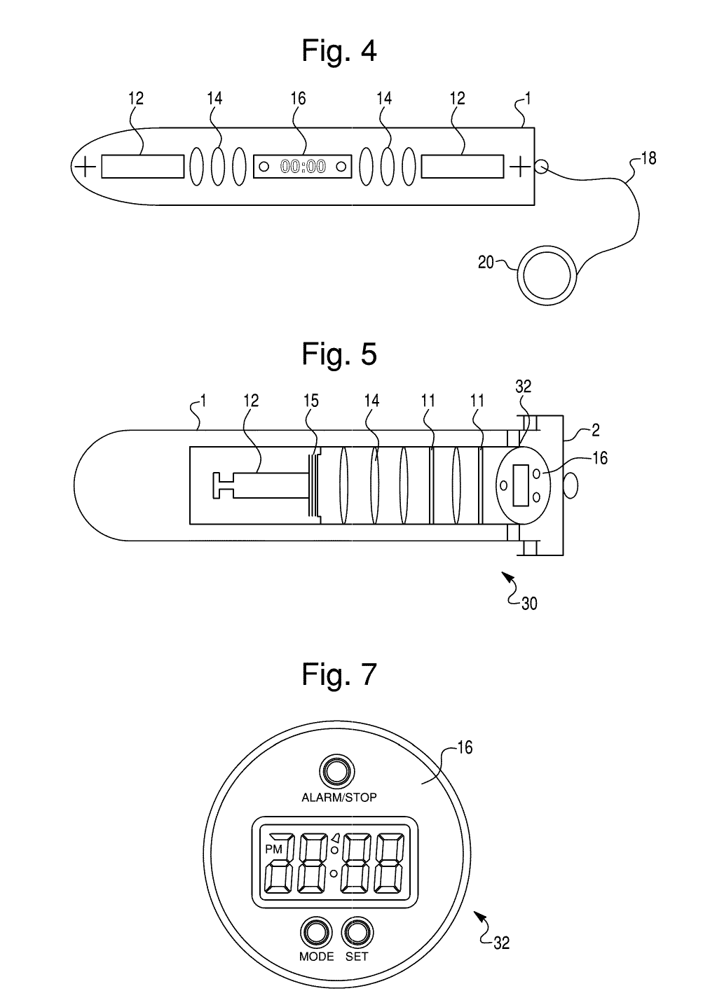

FIGS. 2 and 4 show schematics of the vibrator housing 1 and contents. The housing 1 contains the power source batteries 14, the connected motor 12, the side mounted alarm clock 16. The housing may be optionally equipped with a lanyard/string 18 and ring to aid in the use (retrieval following insertion) and storage of the device. As shown, the device, FIG. 4, can include more than one motor and/or battery source.

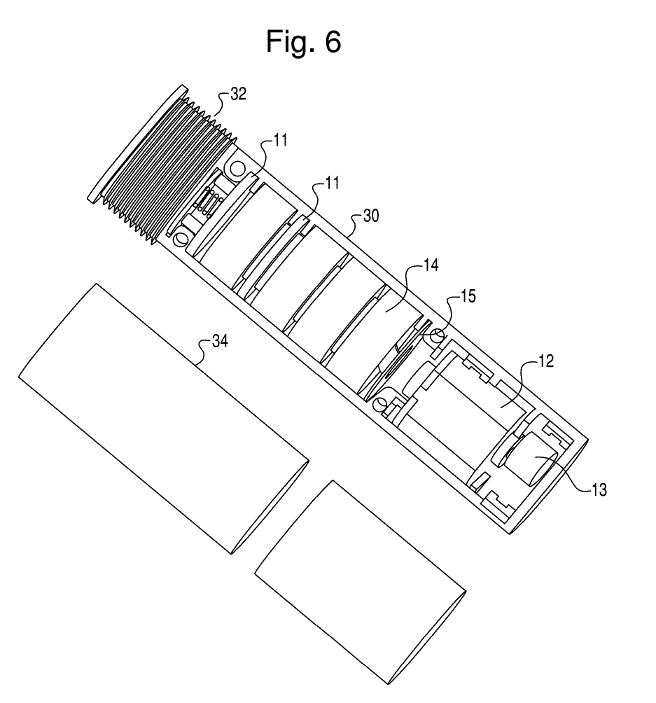

FIG. 5 shows an internal schematic of a vibrator housing 1 including the elements making up the present invention. The housing 1 is open at the right end and closed at the left end by cap 2 as shown in FIG. 5. The housing 1 encloses an inserted cylinder shaped plastic sheath 30 (as shown in FIG. 6) that itself houses the battery 14, motor 12, printed circuit boards 11, battery spring 15. The sheath 30 includes an upper portion 32 that also has the alarm clock 16 integrated therein (an end view is shown in FIG. 7). In use, a user closes the covers 34 onto sheath 30, sets the alarm 16, slides the sheath 30 into the open end of housing 1 and, after sliding it inside the housing, the user will rotate sheath 30 so as to retain the sheath securely in the housing 1 using the threads located on upper portion 32. The housing is then closed by cap 2. A user can set, reset, the alarm function by removing cap 2 and gaining access to the alarm function controls contained on the end of the sheath.

The Pcbs 11 control the motor operation and alarm operation switching functions. If the Pcbs 11 are not included, the clock/alarm function can be self-contained with its own power source, and the motor control of the vibrator can simply be controlled by securing the screw on cap 32 in position. When the spring 15 establishes sufficient electrical continuity, between the batteries and motor leads, the motor will function until spring pressure is released by unscrewing cap 32. Preferably, the motor will function only by setting up the alarm/reminder. Once activated, the motor vibrator will vibrate for a predetermined period of time, i.e., 7, 10, 15, or 20 straight minutes. The user can turn off the vibrating function by opening the top part of the housing 2 and pushing the stop alarm button placed on end portion 32 which will stop the vibrating.

The cap 32 may be self-contained in terms of power, i.e., having a watch battery dedicated to its operation, or it can rely on power from a battery within the battery pack contained within the sheath 30. The primary alarm or reminder function of the device is in the form of initiating operation of the vibrator function. This initiation of the vibrator function may optionally be supplemented by a voice record, music clip, chime, etc. The cap need only be further equipped with a memory chip and playback function in the same way as a “memory” pen is equipped wherein short messages can be recorded for later playback.

In use, a person sets a predetermined time when use of the device is likely to be possible or desirable. The device is then placed into a user's pre-selected bodily cavity. When the alarm/reminder function activates, the user should use the device as soon thereafter as possible inasmuch as it is presently set to run continuously for approximately 7 minutes, for example. The use creates a pleasurable experience and releases certain chemicals within the user that enhances mood, develops relaxation, etc. Once this pattern of use is established, an association is developed between the simple vibrating/audible reminder and a mood/desire for sexual activity. In the same way as certain smells elicit hunger, i.e., passing a bakery or popcorn stand, or a desire for a certain drink, i.e., passing a coffee shop, the alarm/reminder function of this device triggers association with past pleasure/chemical release. Henceforth, the device itself can instigate and “set” the mood and necessary intent for sexual activity simply by vibrating with or without an accompanying sound. In this way, with patterned use, an individual and/or couple can maintain a healthy sexual relationship without waiting for a mood or mindset to occur. Rather, the device itself, through use and association, instigates the necessary mindset.

While the present invention has been described above in terms of specific embodiments, it is to be understood that the invention is not limited to these disclosed embodiments. Many modifications and other embodiments of the invention will come to mind of those skilled in the art to which this invention pertains, and which are intended to be and are covered by both this disclosure and the appended claims It is indeed intended that the scope of the invention should be determined by proper interpretation and construction of the appended claims and their legal equivalents, as understood by those of skill in the art relying upon the disclosure in this specification and the attached drawings.

I claim:

1. A device used for sexual stimulation, comprising:
a hollow longitudinal housing open at one end and closed at a second end, and a cap for closing said open end;
a motor contained within said housing for creating a vibration when activated;
a power source also contained within said housing to provide electric power to activate said motor;
a control system for selectively activating and deactivating said motor; and,
a clock and associated alarm system connected to said control system, said alarm system including a setting function associated with said clock for providing an automatic initiation of said control system as a reminder to a user that use of said device should be imminent.

2. A device as in claim 1, further comprising:
a replaceable cover for wrapping around an outer portion of said housing.

3. A method of maintaining a desired level of sexual activity for an individual, comprising the steps of:
pre-determining a time when sexual activity will likely be appropriate for that individual;
setting a reminder function into an alarm clock, mounted to a device used for sexual stimulation using a vibration function, so as to control initiation of said vibration function at a time corresponding to said predetermined time;
inserting said device into a pre-selected bodily cavity of said user;
waiting for said alarm clock to activate said vibration function; and, thereafter;
engaging in sexual activity using said device as soon after said alarm function has initiated said vibration function.
